(19) United States
(12) Patent Application Publication
(10) Pub. No.: US 2006/0263809 A1
(43) Pub. Date: Nov. 23, 2006

(54) Method for sensitive measure of low level apoptosis in cells

(75) Inventors: Judith Campisi, Berkeley, CA (US); Joshua Goldstein, San Diego, CA (US)

Correspondence Address: LAWRENCE BERKELEY NATIONAL LABORATORY, ONE CYCLOTRON ROAD, MAIL STOP 90B, UNIVERSITY OF CALIFORNIA, BERKELEY, CA, 94720, US

(73) Assignee: The Rgents of The University of California

(21) Appl. No.: 11/399,120

(22) Filed: Apr. 5, 2006

Related U.S. Application Data
(60) Provisional application No. 60/668,895, filed on Apr. 5, 2005.

(51) Int. Cl. C12Q 1/68; G01N 33/574; A61K 38/04

(52) U.S. Cl. 435/6; 435/7.23; 514/19

(57) ABSTRACT

Herein is described a method of detecting apoptotic cells and monitoring apoptosis in a caspase-independent manner. Cells are treated with a caspase inhibitor, and then a marker of apoptosis, such as a caspase-independent signaling protein, is detected for an extended period of time. When cells were suspended in the process of apoptosis and scored for apoptotic cells, the method was shown to be more sensitive than conventional assays. When apoptosis was induced by certain inhibitors, the method is capable of measuring cumulative background levels of apoptosis over a multi-day interval.

### CROSS-REFERENCE TO RELATED APPLICATIONS

This application claims priority to U.S. Provisional Patent Application No. 60/668,895, filed on Apr. 5, 2005, and is hereby incorporated by reference in its entirety.

### STATEMENT OF GOVERNMENTAL SUPPORT

This invention was made during work supported by National Institutes of Health fellowship (AG24015) and grant (AG17242), Department of Defense grant DAMD17-02-1-0443, and the U.S. Department of Energy under Contract No. DE-AC02-05CH11231. The government has certain rights in this invention.

### BACKGROUND OF THE INVENTION

1. Field of the Invention

The present invention relates assays for measuring apoptosis and cell death in cells.

2. Related Art

Apoptosis, or programmed cell death, is the process by which damaged, dysfunctional, virally infected, or potentially neoplastic cells are actively eliminated from multicellular organisms. Apoptosis can be initiated by a variety of physiological and environmental signals. In all cases, the signals engage an intrinsic program of biochemical events that culminates in the enzymatic degradation of key cellular constituents, including regulatory and structural proteins and genomic DNA. The remaining cell components are sequestered into membrane bound bodies and engulfed by neighboring cells or macrophages. Thus, apoptosis provides a means to purge tissues of unwanted or defective cells without the destructive reactions of autolysis, inflammation or autoimmunity (Walker et al., 1988).

Apoptosis is also essential for normal metazoan embryogenesis, during which it eliminates excess cells or cells that fail to make proper functional connections. In complex organisms such as mammals, apoptosis is also vital for the peri- and post-natal maturation of certain tissues, particularly the immune system. In such organisms, apoptosis is additionally important for the homeostatic maintenance of certain tissues during adult life, where cell proliferation and death are balanced to ensure proper tissue size, architecture and function. In these adult tissues, apoptosis is especially important for eliminating cells at risk for neoplastic transformation (Green and Evan, 2002; Jacobson et al., 1997; Meier et al., 2000; Vaux and Korsmeyer, 1999).

Both deficits and surfeits in apoptosis can cause or contribute to pathology, particularly the hyperproliferative and degenerative pathologies associated with mammalian aging (Campisi, 2003a; Campisi, 2003b). It is well-established that defects in apoptosis promote the development of cancer (Green and Evan, 2002). The finding that the apoptotic response to genotoxic stress declines with age (Suh et al., 2002) may in part explain the sharp rise cancer incidence that occurs during aging. On the other hand, excess apoptosis, and the consequent depletion of proliferative precursor or stem cell pools, may contribute to age-related loss of tissue function (Jejurikar and Kuzon, 2003; Nishimura et al., 2005; Sharpless and DePinho, 2004; Tyner et al., 2002). For example, apoptosis was elevated in the crypts of small intestinal villi in mice that age prematurely owing to germline ablation of genes encoding the telomerase catalytic component and WRN DNA helicase/exonuclease (Chang et al., 2004). Cells cultured from other mouse models of accelerated aging also showed increased susceptibility to apoptosis (de Boer et al., 2002), whereas cells from long-lived mouse strains were resistant to (likely apoptotic) cell death (Murakami et al., 2003).

If deficits or surfeits in apoptosis contribute to mammalian aging, changes in the rate of apoptosis most likely take place slowly and cumulatively over the adult life span (e.g., over many weeks or months in the case of mice, or many years in the case of humans) (Hasty et al., 2003). Apoptosis is a rapid event, and essentially undetectable once complete. Thus, under most circumstances, the number of cells undergoing apoptosis in a given tissue at a given time is likely to be small. Consequently, small changes in the incidence or rate of apoptosis are difficult to measure. These considerations raise the possibility that the role of apoptosis in aging has been underreported, both in vivo and cell culture models, due to the technical challenge of detection.

Cell death can broadly be divided into programmed cell death and disordered cell death. In programmed cell death, (apoptosis, autophagy, there may be others) cells activate an enzymatic program that kills the cell in a specific way. In the case of apoptosis, key cellular components such as DNA are chopped up into little pieces and the cell externalizes phosphatidlyserine, which is a signal for neighboring cells to engulf the remnants of the apoptotic cell. In this way apoptotic cells are removed before they leak their contents and alert the immune system which could induce inflammation.

Besides ordered cell death, there is necrotic, or disordered cell death. Necrosis is harder to biochemically define because there are nearly infinite ways of stressing cells so that they fall apart in a disordered fashion. Experimentally, the definition of necrosis is loss of cell membrane integrity. If the cell membrane is broken, then the cell is dead. Also, DNA dyes, such as propidium iodide (PI) which are normally excluded from the cell, can get in and stain the DNA. This make a convenient assay and PI can be used to measure necrosis.

In the body, apoptotic cells get phagacytosed by neighboring cells (or macrophages) and should never become PI positive. In cell culture, however, most cells do not phagacytose their apoptotic neighbors very well (especially when all the cells have been treated with the same apoptosis-inducing agents.) This means that the apoptotic cells sit around until their cell membrane breaks, at which point they become PI positive and by definition necrotic. Since they have already gone though the process of apoptosis (chopped DNA and cytoskeleton, mitochondrial membrane potential loss, externalized PS, etc.), it is considered secondary necrosis.

Apoptosis is identified by both morphological and biochemical changes, including loss of mitochondrial membrane potential (ΔΨm), externalization of plasma membrane phospholipid phosphatidlyserine, DNA fragmentation and nuclear condensation, and, for adherent cells, detachment from the substratum. These changes result from the activation of caspases, a family of cysteine proteinases (Wolf and Green, 1999). Caspases cleave a number of substrates to initiate or effect many of the changes associated with apoptosis. These substrates include the Inhibitor of Caspase-activated Deoxyribonuclease (ICAD), cleavage of which initiates DNA fragmentation (Enari et al., 1998), Acinus, processing of which causes chromatin condensation (Sahara et al., 1999), and a subunit of electron transport chain complex I, cleavage of which initiates the drop in ΔΨm (Ricci et al., 2004).

Conventional apoptosis assays typically measure a specific aspect of the apoptotic process. For example, apoptotic cells in which caspases have been activated contain encapsulated fragmented DNA, which, after cell permeabilization, can be detected by fluorescence flow cytometry as bodies containing less than a G1 DNA content (sub-G1 DNA content assay) (Ormerod et al., 1992). Cells with activated caspases also become permeable to the DNA binding dye propidium iodide (PI) (Cotter and Martin, 1996), scramble phosphatidylserine from the inner to outer plasma membrane leaflet (Koopman et al., 1994; Martin et al., 1995), and lose ΔΨm (Ricci et al., 2004). These events are commonly detected in single cells by fluorescence flow cytometery or microscopy. Thus, caspases are responsible for many of the markers of apoptosis that form the basis for current assays.

Caspases are activated by two major pathways, termed endogenous and exogenous. The endogenous pathway is generally engaged by cell damage or stress, and is characterized by the dominant role of mitochondria. Activation of this pathway results in the translocation of Cytochrome c and other proteins from the mitochondrial inter-membrane space to the cytosol and subsequent drop in ΔΨm. Once in the cytoplasm, Cytochrome c interacts with the adaptor protein Apaf-1, which recruits and activates caspases in a complex termed the apoptosome (Green and Evan, 2002). Thus, signaling events that trigger the endogenous pathway often occur prior to caspase activation and therefore are caspase-independent.

The exogenous pathway, by contrast, activates caspases though cell surface death receptors. Upon binding physiological ligands, these receptors activate upstream caspases, which in turn either activate the caspases that cause the apoptotic phenotype, or cleave proteins that initiate the endogenous pathway (Ashkenazi and Dixit, 1998). Effector caspases, once activated, will cause the degradation or collapse of DNA, the nuclear lamina, the cytoskeleton, and other critical components of cellular integrity. Thus, Cytochrome c release depends on caspase activation by the exogenous pathway, but not the endogenous pathway.

Caspases recognize four amino acid motifs, the fourth of which is invariably aspartate. Tri-peptide caspase inhibitors such as benzyloxycarbonyl-Val-Ala-Asp-fluoromethylketone (zVAD) and carboxyterminal o-phenoxy group conjugated to Glu-Val-Asp (QVD) generally bind the active sites of all caspases, despite the fact that individual caspases have different four amino acid peptide specificities. These inhibitors bind caspases irreversibly, thereby blocking caspase-dependent apoptotic events (Caserta et al., 2003; Thomberry and Lazebnik, 1998).

Background or basal rates of apoptosis likely change during mammalian aging (Suh et al., 2002). Such changes may be physiologically relevant to the loss of tissue structure and function that is a hallmark of aging, and may also contribute to both the hyperplastic and degenerative diseases of aging (Campisi, 2003b; Joaquin and Gollapudi, 2001; Zhang and Herman, 2002). However, rare apoptotic events are difficult to quantify. Herein, we describe a new apoptosis assay that is more sensitive than conventional assays for detecting basal or low levels of apoptosis in cells.

### SUMMARY OF THE INVENTION

Age-associated loss of tissue function and several chronic diseases are thought to derive in part from the cumulative effects of subtle changes in the level of apoptotic cell death. Because apoptosis is rapid and essentially undetectable once complete, small changes in its incidence are difficult to detect, even in well-controlled cell culture models.

Herein is described a new apoptosis assay that provides greater sensitivity than conventional assays because it measures the accumulation of apoptotic cells. Cells that initiate apoptosis are preserved for an extended period of time by inhibiting caspase activity. The present invention provides a method of detecting and monitoring apoptosis in cells comprising the following steps: (1) providing a cell sample, (2) treating the cells with a caspase inhibitor for an extended period of time, (3) washing the cells, (4) treating the cells with an antibody to a caspase-independent signaling protein to stain apoptotic cells and (5) detecting apoptotic cells.

In one aspect of the invention, the method further comprises the step of treating the cells with a stain or dye to detect a marker of apoptosis such as, chromatin condensation, phosphatidylserine exposure or nuclear fragmentation.

In another aspect of the invention, the caspase inhibitor is a general caspase inhibitor or a specific inhibitor to Caspase 1, 2, 3, 4, 5, 6, 7, 8, 9, 10 or 13. In a preferred embodiment, the caspase inhibitor is selected from the group consisting of Quinoline-Val-Asp-Ch₂-O-Ph (QVD), zVAD or BAF.

In another aspect, the extended period of time is at least 1, 2, 3, 5 or 7 days, more preferably at least 3 days for most cell types. In some embodiments, the period of time is at least 7 days.

In yet another aspect, the caspase-independent signaling protein that is used to monitor apoptosis is a pro-apoptotic Bcl-2 family member, including Bax, Bad, Bim, Puma, Noxa; or a mitochondrial protein that is caspase-independently released during apoptosis, including but not limited to, Cytochrome c, SMAC/Diablo, Omi/HtrA2, endonucleaseG, and apoptosis inducing factor (AIF).

In one aspect, the cells are treated with an antibody against the caspase-independent signaling protein. In one embodiment, a secondary antibody is used to increase the signal of the antibody bound to the caspase-independent signaling protein. In a preferred embodiment, the secondary antibody is conjugated to a fluorescent probe to be used for detection.

In another aspect, the apoptotic cells are detected to determine number or percentage of cells in the cell sample undergoing apoptosis. Mitochondrial proteins localization would follow the pattern of Cytochrome c, while the localization pattern of useful pro-apoptotic Bcl-2 family member proteins would be the opposite. Anti-apoptotic Bcl-2 family members do not move during apoptosis, but some, but not all pro-apoptotic Bcl-2 family members move. The members that do not move (i.e., Bak) would not be useful for this assay.

When cells were suspended in the process of apoptosis and scored by immunostaining cytochrome c, which is redistributed from mitochondria in healthy cells to the cytoplasm in dying cells, the method was shown to be more sensitive than conventional assays, including sub-G1 peak, mitochondrial membrane potential loss, and propidum iodide exclusion assays. When apoptosis was induced by actinomycin D, the method was capable of measuring cumulative background levels of apoptosis over a multi-day interval.

Using this assay, normal fibroblasts undergo very low levels of apoptosis upon X-irradiation, indicating dominance of the senescence response in this cell type. Further, it is shown that apoptosis increases subtly but measurably when human mammary epithelial cells enter crisis, a state that can lead to replicative immortality, indicating that cell death that occurs at crisis is largely non-apoptotic.

### BRIEF DESCRIPTION OF THE FIGURES

FIG. 1 shows a panel of photographs showing that QVD preserves adhesion of Act-D-treated cells. MEFs were given Act-D (40 nM in DMSO) or DMSO and either no inhibitor, zVAD (100 μM) or QVD (40 μM). After 3 days, the cells were photographed at 10× magnification.

FIG. 2 is a panel of graphs showing a comparison of apoptosis assays. Sub-G1 peak (A), mitochondrial membrane potential (B), and propidium iodide exclusion (C) assays were performed using MEFs treated with Act-D or DMSO in the presence or absence of QVD. The cells were assayed every 24 hours for 3 consecutive days. Shown is one of two experiments that gave similar results. A) Sub-G1 peak FACS measurements. M1 (left part of the histogram) shows the gate that detects apoptotic cells in cultures treated with DMSO (Top) or Act-D (Bottom) after 3 days. A time course of the fraction of cells in the M1 compartment is shown on the right. B) FACS analysis of mitochondrial membrane potential in living cells. M1 (left) shows the distribution of cells with low membrane potential in DMSO-(Top) or Act-D-treated (Bottom) cultures after 2 days. The time course of fraction of cells the M1 compartment is on the right. C) FACs analysis of propidium iodide exclusion. M1 (right) shows distribution that fail to exclude PI in DMSO-(Top) and Act-D-treated (Bottom) cultures. The time course is shown on the right.

FIG. 3 is a graph and photographs of the results of the CICR assay. A. Cumulative apoptosis induced by Act-D or normal culture conditions (basal apoptosis). MEFs were scored for diffuse cytochrome c after treatment with DMSO or Act-D for 1-3 days in the presence or absence of QVD. Images (Left, 20×) show representative cells after 1 day. The arrows show diffuse cytochrome c staining (Left, top) and nuclear staining (Left, bottom). A time-course of cells with diffuse cytochrome c staining is shown on the right. Approximately 20-750 cells were counted per condition. Error bars represent SD, n=4 fields. B. CICR measures basal apoptosis over a 3 day interval. MEFs were cultured in 3% oxygen in the presence of QVD for 3 days and assayed daily for apoptotic cells using the CICR assay. Shown are two independent experiments, which scored between 600-800 cells per condition.

FIG. 4 is a graph and photographs showing the measurement of apoptosis by CICR in X-irradiated human fibroblasts. Proliferating WI-38 fibroblasts were untreated (Control) or X-irradiated with 10 Gy (X-ray) and assayed for 3 days by CICR. Arrow shows an apoptotic cell identified by diffuse cytochrome c staining (left, top) and the corresponding nucleus (left, bottom). Quantification of Control (362) and X-irradiated (428) cells is shown on the right. * p=0.049 in an unpaired single tailed Student t-Test, n=8 fields.

FIG. 5 graphs and photographs show that the CICR assay detects apoptosis in HMECs in crisis. HMECs were cultured to the end of their replicative life span (agonescence/senescence) and induced into crisis by at p53 inactivation (HMECs) or p53 inactivation and E7 expression (HCA-2). The cells were then assessed for apoptosis by CICR for a 3-day period. Arrows identify apoptotic cells (diffuse cytochrome c staining, bottom). The number of cells scored were 247 (agonescence, Agn), 465 (HMEC crisis, Crs), 343 (replicative senescence, Sen), and 484 (HCA-2 crisis, Crs). *p=0.01 and **p=0.02 in an unpaired single tailed Student t-Tests, n=5 fields.

FIGS. 6A and 61B are photographs of the cells after the apoptosis assay. FIG. 6A. Immunocytochemistry. HeLa cells were treated with vehicle (DMSO Control), actinomycin D (ActD) to induce apoptosis, or ActD plus the caspase inhibitor ZVAD-fmk. 24 h later, cells were immunostained for cytochrome c (green) and counterstained with DAPI (blue) to identify nuclei. In control cells, cytochrome c is evident as punctate mitochondrial staining. After ActD treatment, fewer cells are present on the culture dish owing to apoptosis. After ActD+ZVAD treatment, most cells remain on the dish, but show diffuse cytoplasmic cytochrome c staining. FIG. 6B. Quantification. The % cells with diffuse cytochrome c staining (x axis) were scored 24 h after treatment with vehicle (Contr), ActD or ActD plus ZVAD (ZV). Cytochrome c staining in the presence of ZVAD allows apoptotic cells to accumulate over 24 h.

FIG. 7 is a graph showing QVD does not inhibit growth. HCA-2 fibroblasts and HMEC were grown in normal growth media (DMEM, MEGM) or normal growth media supplemented with 0.1% DMSO (DMSO) or 0.1% DMSO+40 μM QVD (QVD). HCA-2 growth curves were averaged triplicates (HCA-2) or duplicates (HMEC) and error bars (HCA-2) are SD.

FIG. 8 is a bar graph and photographs of cells showing attachment of cells with diffuse cytochrome c in the presence of QVD. A. MEFs and HeLa cells were grown in media containing Act-D (MEFs 40 nM, HeLa 200 nM) and QVD for 8 days. Media and drugs were replaced daily. Each data point is the average of four cells counts and error bars are SD. B. A CICR analysis of HeLa cells treated with Act-D. HeLa were scored for diffuse cytochrome c after treatment with Act-D (200 nM) for 0-3 days in the presence QVD. Images (Left, 20×) show cytochrome c (top) and nuclei (bottom) after 1 day. Arrows mark cells that have not released cytochrome c. Quantification is on the right. n=780 (Day 0), 790 (Day 1), 410 (Day 2), and 251 (Day 3) cells.

### DETAILED DESCRIPTION OF THE PREFERRED EMBODIMENT

The present invention provides a method based upon the caspase-independent endogenous apoptotic pathway to develop a sensitive assay that can measure the number of apoptotic cells that accumulate over a multi-day interval. Using conventional or known methods, rare apoptotic events are difficult to quantify. The present method uses caspase inhibitors to essentially freeze cells in the apoptotic process after mitochondrial cytochrome c release but before destruction of the cells. This caspase-independent cytochrome c release (CICR) method can detect small changes in the incidence of apoptosis in cells and cell culture models. By doing so, it should help facilitate our understanding of how chronic cellular damage or stress can influence aging phenotypes by subtly altering the apoptotic response.

It is further contemplated that the use of this assay ex vivo and in vivo for a sensitive and useful for measuring rare apoptotic events in animals and humans.

In addition, the CICR assay is more sensitive than the conventional assays of sub-G1 DNA content, mitochondrial membrane potential measurements, and loss of plasma membrane integrity (PI exclusion). However, the CICR assay will not monitor apoptotic events that do not engage the mitochondria, such as those that signal through the exogenous apoptosis pathway. Nonetheless, the CICR assay successfully measured the background levels of apoptosis in MEF cultures and the low levels of apoptosis that occur in X-irradiated human fibroblast cultures HMEC cultures at agonescence and crisis.

Caspases are known for being responsible for many of the markers of apoptosis that form the basis for current assays. Caspase inhibitors, such as QVD, zVAD, BAF and others, block apoptosis, at least temporarily, thereby allowing cells to remain attached until other forms of cell death occur. The present method combines the use of caspase inhibitors and cytochrome c immunocytochemistry to provide an assay that permits the accumulation of cells caught in the act of apoptosis over an extended interval and their detection. In one embodiment, this assay is a caspase-independent cytochrome c release (CICR) assay.

In one embodiment, the method of detecting and monitoring apoptosis is comprised of the following steps: (1) providing a cell sample, (2) treating the cells with a caspase inhibitor for an extended period of time, (3) washing the cells, (4) treating the cells with an antibody to a caspase-independent signaling protein to stain apoptotic cells and (5) detecting apoptotic cells.

Cell samples can be comprised of cells from human, other mammalian, and vertebrate organisms. In a preferred embodiment, the cell samples are tissue samples, plasma, human cells, including but not limited to epithelial cells, fibroblasts, macrophages, chandrocytes, muscle cells, neurons, mammary, gastrointestinal cells and stem cells. Types of epithelial cells that can be used include, but are not limited to, mammary, lung, liver, kidney, prostate, pancreas, ovary, testes, uterus, intestine (colon, small intestine, large intestine) stomach, esophagus, skin, mouth (larynx, pharynx) as well as other cell lines from other tissues. Epithelial cell types that have been tested and are appropriate for use in this invention included human mammary epithelial cells and cell lines that are immortalized or tumorigenic. Multiple cell types can be present in the cell sample.

The cell samples are plated or grown in a container. In a preferred embodiment, the cells are grown in each well of a Lab-Tek 4 well chamber slide (Nalge Nunc International, Naperville, Ill.) or similar container. In all cases, growth media appropriate to the cell type, containing necessary proteins, vitamins and other additives, such as growth factors, pituitary extract, insulin, and hydrocortisone, may be added to the cells when needed to promote and maintain cell growth and function.

The cell sample is treated with a caspase inhibitor, such as Quinoline-Val-Asp-Ch₂-O-Ph (QVD), benzyloxycarbonyl-Val-Ala-Asp-fluoromethylketone (zVAD), or boc-aspartyl-fluoromethylketone (BAF), for several to many days. Any general caspase inhibitor or specific inhibitor to Caspase 1, 2, 3, 4, 5, 6, 7, 8, 9, 10 and 13, is contemplated as being useful in this method. Such inhibitors are known to those having skill in the art and are commercially available. The caspase inhibitors can be used singly or in combination.

The caspase inhibitor concentration can be varied and dependent on cell type or other experimental parameters. To find a suitable starting concentration, the inhibitor concentration can be titrated down to the lowest concentration wherein the cells in the cell sample appear to be undergoing apoptosis. This concentration is then multiplied by a factor of 2. In a preferred embodiment, the concentration of the caspase inhibitor is between 30-50 μM, more preferably about 40 μM.

This method requires a measurable caspase-independent signal (e.g. Cytochrome c localization in a cell) which allows the inhibition of caspases, which in turn allows us to preserve and accumulate apoptotic cells. The preservation and accumulation of apoptotic cells should boost the sensitivity of the assay. Thus any measurable caspase-independent signal used as a metric must both change measurably during apoptosis and do this in a caspase-independent manner. In one embodiment, the assay is based on the number of cells that have released cytochrome c from the mitochondria to the cytosol.

A major strength of the present method is its ability to monitor the kinetics of apoptotic events over a multi-day period. In a preferred embodiment, the accumulation of cells to monitor apoptotic events is performed over at least 1, 2, 3, 5 or 7 days, more preferably at least three days. However some fragile cell types may not survive for at least three days. The goal is to monitor and accumulate cells for an extended period of time to obtain the most accurate measurement. Since some tumor cell lines can survive with cytosolic cytochrome c for an extended period of time, in another embodiment, the accumulation of cells can be monitored for at least one week.

At the end of the extended period of time, the cells are washed with buffers, such as PBS, to remove any proteins that were in the media, such as serum albumin that might non-specifically bind antibodies. Immunostaining protocols routinely employ blocking buffers to block non-specific interactions to antibodies. In our case, the blocking buffer also permeabilizes the cell membrane to allow the antibodies to enter the cell for staining cell structures.

Cells are then incubated with an antibody against a caspase-independent signaling protein in the apoptotic pathway to stain apoptotic cells. The signal from the antibody can also be increased by secondary antibody staining as is known and practiced in the art. Immunostaining protocols typically used secondary antibody staining to increase and amplify the hybridization signal. In a preferred embodiment, the secondary antibody is conjugated to a fluorescent probe to permit the viewing of cells with a fluorescent microscope. There are many fluorescent DNA dyes that can be used for counting cells as is known in the art (for example, see Molecular Probes (URL:<http://www.probes.com>).

In a preferred embodiment, an anti-Cytochrome c antibody is used to stain apoptotic cells. In another embodiment, an antibody to any mitochondrial protein that is caspase-independently released during apoptosis, including but not limited to, Cytochrome c, SMAC/Diablo, Omi/HtrA2, endonucleaseG, and apoptosis inducing factor (AIF) is used to stain apoptotic cells. In another embodiment, some of the pro-apoptotic Bcl-2 family members might also serve as the caspase-independent metric for apoptosis. For example, a Bax antibody could measure apoptosis induced by certain pathways (in specific cells). Additionally some of the other Bcl-2 family members might translocate to the mitochondria in a caspase-independent manner. This list would include most of the “BH3 only” pro-apoptotic members such as Bad, Bim, Puma, Noxa, and others.

One issue with using the location in the apoptotic pathway of pro-apoptotic Bcl-2 proteins as a measure is that they are only activated as a subset of physiological death stimuli and are therefore less general than Cytochrome c. Thus, if there are commercially available antibodies to measure their cellular location in the later steps of the method, pro-apoptotic proteins may be useful in this method. For example, a well known antibody to the active form of Bax would more general than antibodies to “BH3 only” proteins, however less general than that of Cytochrome C.

Once released from mitochondria, Cytochrome c (Cyt c) binds to the protein, apoptosis protease activating factor-1 (APAF-1) complexed with dATP and forms the oligomeric apoptosome complex. Therefore, in a preferred embodiment, any antibody made to an active form of APAF-1 would serve as a good caspase-independent metric for apoptosis. APAF-1 could perform as well as or better than cytochrome c as an indicator of apoptosis.

In another embodiment, other mitochondrial proteins, that are released during apoptosis concurrently with Cytochrome c, could also be used as a metric for measuring apoptotic cells by antibody binding. These mitochondrial proteins may include, but are not limited to: SMAC/Diablo, Omi/HtrA2, endonucleaseG, and apoptosis inducing factor (AIF). These proteins would have a pattern of localization similar to that of Cyt c. Mitochondrial proteins localization would follow the pattern of Cytochrome c, while the localization pattern of useful pro-apoptotic Bcl-2 family member proteins would be the opposite. Anti-apoptotic Bcl-2 family members do not move during apoptosis, but some, but not all pro-apoptotic Bcl-2 family members move. The members that do not move (i.e., Bak) would not be useful for this assay.

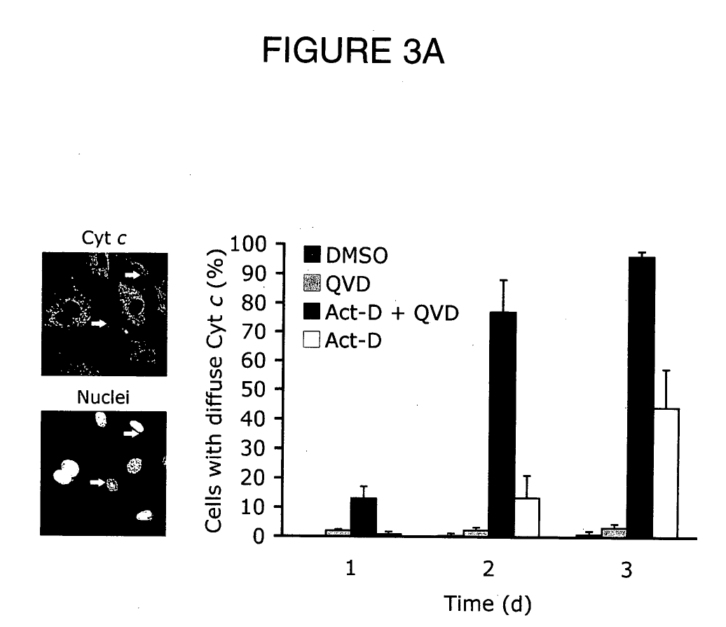

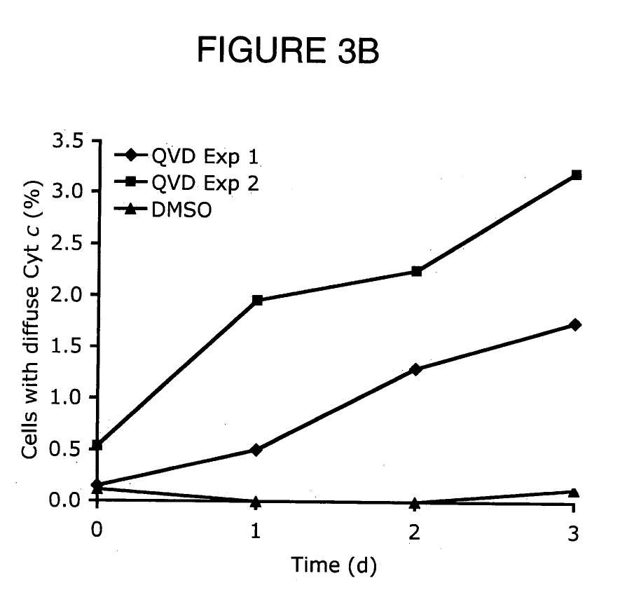

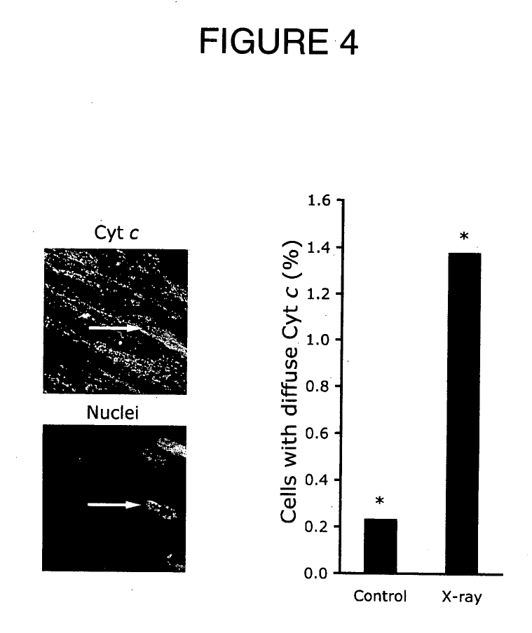

The method can further comprise a step of treating the cells with a stain or dye to detect chromatin condensation, phosphatidylserine exposure or nuclear fragmentation, or other hallmarks or indications of apoptosis for comparison. In a preferred embodiment, chromatin condensation is also detected to distinguish healthy cells from apoptotic cells. Chromatic condensation can be detected using fluorescent DNA-binding dyes or stains such as Hoescht 33342. Since a stain such as Hoescht 33342 penetrates all cells, the combination of these two stains allows for the distinction among healthy cells that stain only with Hoescht (see FIGS. 3 and 4).

It is not expected that the caspase inhibitors, such as QVD, will block necrotic death, so this subset of cells would likely disintegrate or float away and not be counted as apoptotic or healthy. In other words, the present method likely does not distinguish between apoptotic and necrotic death. However, in tissue culture, at the end of the apoptotic process, cells will stain with propidium iodide. This “secondary necrosis” would be detected as apoptotic cell death, unlike many conventional assays. In the present method, caspase inhibitors block downstream apoptosis, which means the cells should take much longer to reach secondary necrosis, at which point the cells will probably float away or disintegrate. In a preferred embodiment, if the cells are scored before they undergo necrosis (e.g. a time period of days to a week), then the cells can be counted as apoptotic (e.g. a localization pattern for apoptotic cells and still present in the sample because they have not floated away or disintegrated).

In the last step of the method, apoptotic cells are detected to determine number or percentage of cells in the cell sample undergoing apoptosis. In a preferred embodiment, cells are scored by observing the pattern of fluorescence of the antibody stained and scored as apoptotic if stained with the antibody against a caspase-independent signaling protein in the apoptotic pathway. In general, the cells are scored using the following criteria: 1) presence of diffuse protein in both the cytoplasm and nucleus; 2) lack of reticulated or punctate staining in the cytoplasm. The localization of pro-apoptotic Bcl-2 family members in apoptosis would have a diffuse pattern in healthy cells and a punctate distribution in apoptotic cells. The localization pattern of mitochondrial proteins in the signaling pathway that are released during apoptosis would have a punctuate distribution in healthy cells and a diffuse pattern in apoptotic cells. These localization patterns may not identify the sub-cellular localization of the apoptosome.

Typically images can be acquired using a low magnification objective (e.g. 20×), and between 10-10,000 cells are scored. In controls where no caspase inhibitor is used, most of the cells may die and float away, thereby decreasing the number of cells that can be scored. In a preferred embodiment, about 300-600 cells are scored. More cells counted increases the statistical significance of the result.

Cells can be scored by counting nuclei using images of uniformly illuminated staining imported into an image viewer. In a preferred embodiment, fluorescent DNA dyes are used to stain and count cells. In other embodiments, other DNA staining methods can be used to score cells. In other embodiments, other methods of staining cells besides staining the cellular DNA can be used, including but not limited to: plasma membrane dyes, cytoskeleton dyes, and staining nearly every other organelle. Cells could be counted by eye through a microscope.

In a preferred embodiment, when using Cytochrome c as the metric for apoptosis, cells are scored as apoptotic if cytochrome c appears diffuse and the stain is evenly distributed within the cell meeting the following criteria: 1) presence of diffuse cytochrome c in both the cytoplasm and nucleus; 2) lack of reticulated or punctate staining in the cytoplasm. Examples of diffuse Cytochrome c are shown in the left side of FIGS. 3A, 4, and 5.

Crisis is a state of genomic instability that occurs both in culture and in vivo (Artandi and DePinho, 2000). Cell populations in crisis are characterized by the simultaneous occurrence of proliferation, cell death and irreversible cell cycle arrest (senescence), generally with little or no net increase, or only a gradual decline, in cell number over time (Hara et al., 1991; Shay et al., 1991). Eventually, cell death predominates over proliferation, and cell populations in crisis generally vanish. Very rarely (especially in human cells), a replicatively immortal variant will arise from populations in crisis, and these variants are orders of magnitude more susceptible to neoplastic transformation (Shay et al., 1993b). Given that crisis poses a great danger for the development of cancer, and that the senescence response is a potent tumor suppressive mechanism (Campisi, 2001), it is not surprising that populations in crisis contain cells that are senescent (Shay and Wright, 2004). We used the present method to show that apoptosis, which is also a potent tumor suppressive mechanism (Shay and Wright, 2004), is similarly upregulated during crisis. The present method was used to detected a significant increase in background apoptosis when we compared HMECs arrested in agonescence with those in crisis. Therefore, in another embodiment, the method can be used to detect cells ex vivo and in vivo that are in crisis.

In one embodiment, the present method can be used to detect apoptosis levels in vivo. One concern in using the method is the toxicity of using caspase inhibitors in vivo. However, according to the technical data sheet for Q-VD-OPH (MP-Biomedicals, Aurora, Ohio), the toxicity of caspase inhibitors should be fairly low. According to the data sheet, there is no toxicity at 1 g/kg of body weight (2 mM). Therefore, it is suggested that for in vivo use of this relatively non-toxic caspase inhibitor, that the concentration be less than 1 g/kg of body weight. In a preferred embodiment, the amount of QVD used of 40 uM should not cause mortality outright because the concentration is about 48 times more dilute than the 1 g/kg sited as safe.

In one embodiment, the present method will aid in explaining a number of biological phenomena that can be modeled in culture and exhibit low but potentially important levels of apoptosis.

In another embodiment, the method can be an ex vivo assay used to test patient biopsy cells to determine, for example, which chemotherapy drug would be most effective in killing cancer cells, i.e. causing apoptosis.

In another embodiment, the method described can be a drug screening and research tool for screening drugs that inhibit or block apoptosis. For example, in a screen for drugs that combat Alzheimer's disease, because neurons and cells undergo apoptosis at greater rate in Alzheimer's, the method would be carried out in the presence of neurons and β-amyloids (which are known to promote Alzheimer's), using a caspase inhibitor such as QVD and the candidate drug. By monitoring the rate of apoptosis, it can be observed whether the drug effectively blocks apoptosis.

In yet another embodiment, the method can be used to screen for compounds that do not induce cell death. For example, the method can be used as an accurate measure of whether a compound would be toxic to cells and induce apoptosis.

In a preferred embodiment, a kit comprising of a set of vials or containers containing the necessary compounds, reagents, inhibitors, antibodies, dyes and buffers formulated and ready for use are provided to carry out the prescribed assay. Sufficient volume of each reagent can be supplied to perform multiple assays in parallel or array format. Complete instructions, formulations should also be supplied with the kit.

### EXAMPLE 1

Because conventional assays provide only a snapshot of the incidence of apoptosis in a cell population, it was reasoned that low levels of apoptosis could be amplified by measuring the number of apoptotic cells that accumulate over a prolonged interval. To test this, cultures of primary mouse embryo fibroblasts (MEFs), normal human neonatal fibroblasts (WI-38), and normal human mammary epithelial cells (HMECs) were used.

Cell culture and reagents: Mouse embryo fibroblasts (MEFs) and human fibroblasts (WI-38) were grown in Dulbecco's Modified Eagle Media supplemented with 10% Fetal Bovine Serum 2 mM L-Glutamine, and 100 units/ml of penicillin and streptomycin. MEFs were derived from C57B16 timed pregnant mice and grown in 3% oxygen as described (Parrinello et al., 2003). WI-38 fibroblasts were purchased from ATCCC. Post-selection human mammary epithelial cells HMECs were cultured in MEGM media (Cambrex, Walkersville, Md.), as described (Hammond et al., 1984). Post-selection HMEC cultures were induced into crisis by inhibition of p53 as described (Stampfer et al., 2003). Act-D, RNase A, bovine serum albumin (BSA), and propidium iodide (PI) were purchased from Sigma-Aldrich. zVAD-FMK and QV-D-OPH were from MP-Biomedicals, Aurora, Ohio. Tetramethylrhodamine ethyl ester (TMRE), Hoechst 33342, and Prolong were purchased from Molecular Probes, Eugene, Oreg.

Subconfluent cultures were treated with the caspase inhibitors zVAD or QVD together with actinomycin D (Act-D; 40 nM), which is known to induce cell death whether or not caspase inhibitors are present. Initially, the cultures were simply scored for cell detachment, a late event in the apoptotic response of adherent cells. As expected, treatment with ActD for 3 days caused all three cell types to adopt characteristic apoptotic morphology and detach from the culture dish.

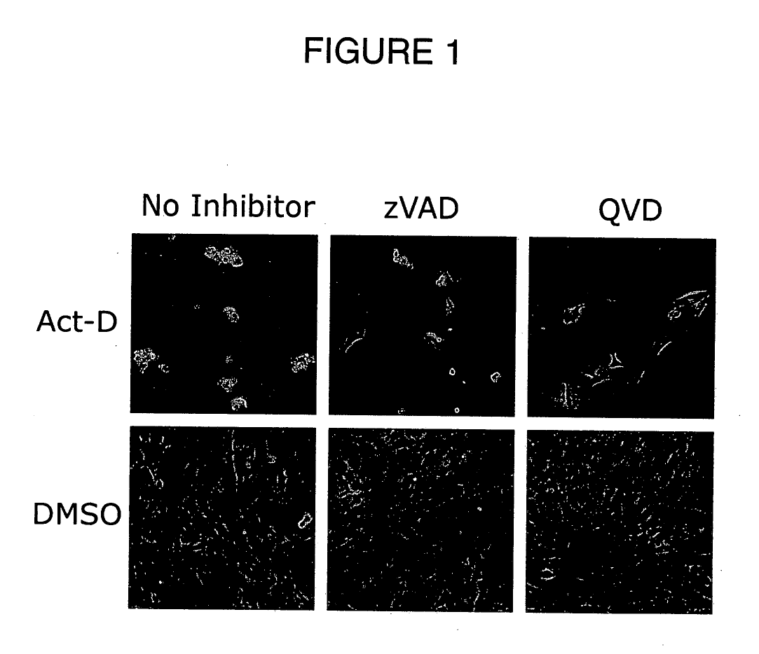

Depending on the cell type, differences were found in the ability of zVAD and QVD to prevent detachment. zVAD, even at 100 μM, failed to prevent detachment in MEFs. However, 40 μM QVD prevented most of the cell detachment under the same conditions (FIG. 1). MEFs treated with Act-D plus QVD remained subconfluent, but, in contrast to control cultures that received the caspase inhibitors but no Act-D, they proliferated (FIG. 1). Act-D treated WI-38 cells, on the other hand, remained equally well attached whether the inhibitor was zVAD or QVD (not shown). In the case of HMECs, a small fraction detached in the presence of Act-D and QVD, but a larger fraction detached in the presence of Act-D and zVAD (not shown). These findings suggested that QVD stalled, at least temporarily, Act-D-induced detachment of MEFs, human fibroblasts and HMECs.

### EXAMPLE 2

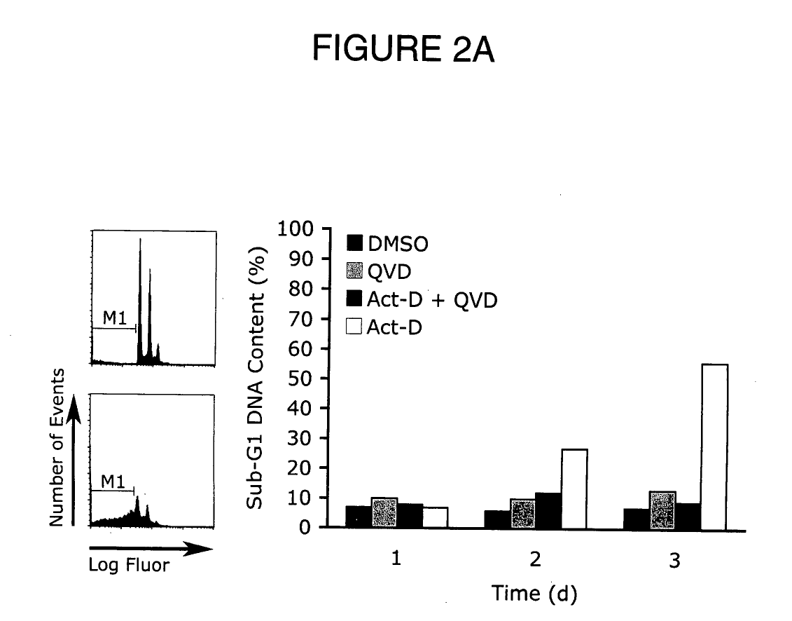

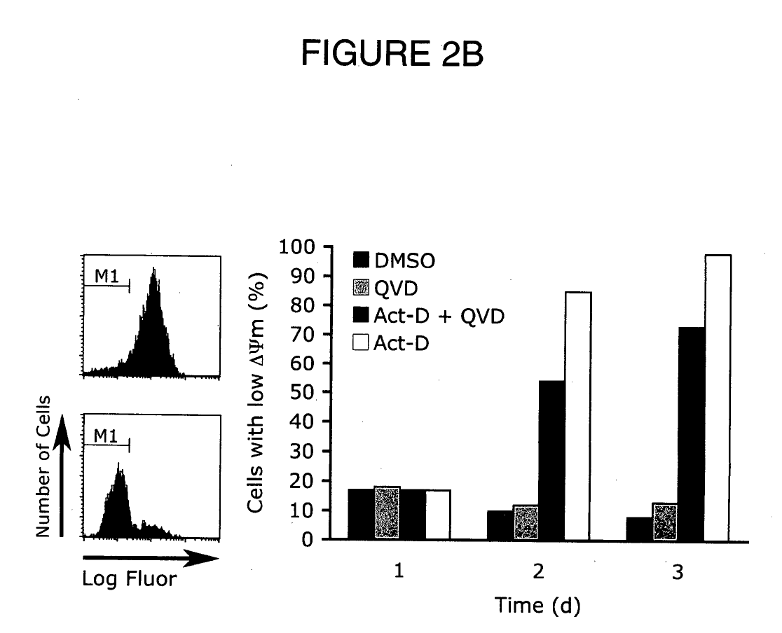

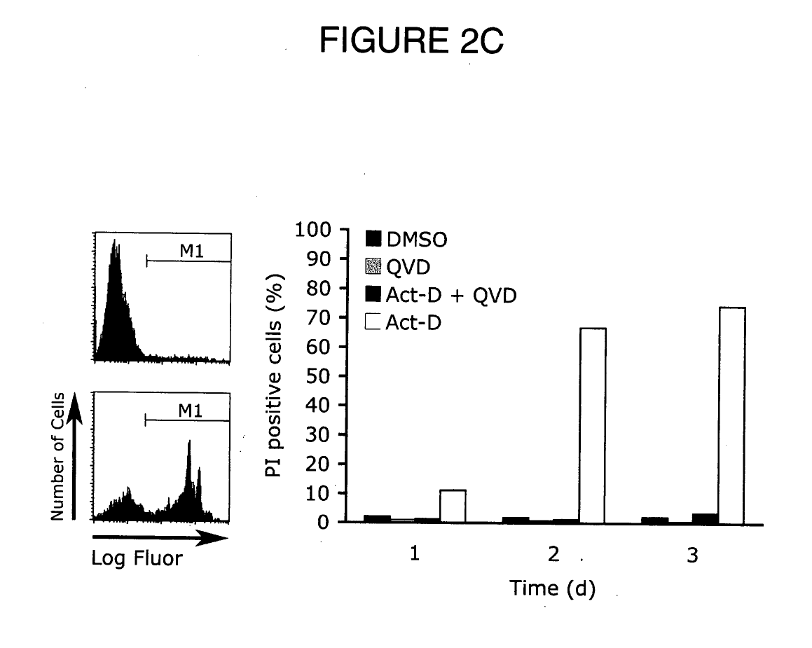

To test this idea more directly, MEFs were treated with Act-D in the absence or presence of QVD and measured apoptosis 3 days later. Three standard assays for apoptosis were used, all of which employ flow cytometry for detection (FIG. 2). These assays were the appearance of a sub-G1 DNA peak (FIG. 2A), reduction in ΔΨm (FIG. 2B), and the ability to exclude propidium iodide and its subsequent binding to nuclear DNA (FIG. 2C). In each case, manifestations of apoptosis were apparent 1-2 days after addition of Act-D and were substantially or completely reduced by the addition of 40 μM QVD.

Cell Death Assays: Sub-G1 peak determination. 1×10⁵ cells were treated with Act-D (40 nM in DMSO), DMSO (0.2%), QVD (40 μM), or QVD plus Act-D for 1, 2, or 3 days. QVD (and Act-D+QVD) was replaced each day. At the end of the experiment, media were collected and washed once with PBS. The PBS was also collected and then the cells were trypisized. Trypsinization was stopped by addition of the previously collected and pooled media and PBS. Cells were then collected by centrifugation at 200 g for 10 minutes, resuspended in 70% ethanol and stored at −20° C. overnight. The fixed cells were resuspended in 300 μl of PBS containing 10 μg/ml PI and 0.5 μg/ml RNase A and incubated at room temperature for 1 hour. Cells were quantified by flow cytometery using a FACscan (Becton Dickinson) in FL-3 and analyzed using Cellquest software (BD) (Cotter and Martin, 1996).

### EXAMPLE 3

Caspase-Independent Cytochrome c Release (CICR) Assay

Act-D-treated MEFs were next examined for the appearance of diffuse cytochrome c immunostaining, indicative of cytochrome c release from the mitochondria (FIG. 3A, Table 2).

Mitochondrial Membrane Potential. 1×10⁵ cells were treated with Act-D and QVD for 1, 2, or 3 days. QVD (and Act-D+QVD) was replaced each day. At the end of the experiment, media were collected and the cells were washed with PBS. The PBS was collected and combined with the media. Cells were trypsinized and resuspended in medium and PBS, as described above. The cells were spun for 5 min at 200 g and resuspended in fresh media containing TMRE (50 nM) and allowed to incubate at 37° C. for 30 minutes. Fluorescence was quantified in FL-2 using a FACscan and analyzed with Cellquest software (Waterhouse et al., 2001).

In contrast to the other indicators of apoptosis, which declined in the presence of QVD, QVD increased the proportion of Act-D treated cells with diffuse cytochrome c staining. Thus, in the presence of QVD, 96% of cells treated with Act-D for 3 days showed evenly distributed cytochrome c immunostaining. In the absence of the inhibitor only a small fraction of the cells remained on the plate. Of those few cells that remained, however, only about 44% showed diffuse cytochrome c immunostaining (FIG. 3A). This result suggests that, in the absence of QVD, apoptosis proceeded until eventual cell detachment and/or disintegration. The result also supports the idea that QVD at least temporarily blocks apoptosis, thereby allowing cells to remain attached until other forms of cell death occur. These observations show that the combined use of QVD and cytochrome c immunocytochemistry might constitute an assay that permits the accumulation and detection of cells caught in the act of apoptosis over an extended interval (at least 3 days). This assay was termed the caspase-independent cytochrome c release (CICR) assay.

Caspase-independent cytochrome c release (CICR) assay. 3×10⁴ cells were plated in each well of a Lab-Tek 4 well chamber slide (Nalge Nunc International, Naperville, Ill.) and treated with Act-D (40 nM), DMSO (0.2%), QVD (40 μM), or QVD plus Act-D for 1, 2, or 3 days. QVD (and Act-D+QVD) was replaced each day. At the end of the experiment, cells were washed twice with PBS and fixed with 4% formaldahyde in PBS for 5 minutes at room temperature. The cells were then washed twice with PBS and then incubated in blocking buffer (3% BSA and 0.1% Triton-X in PBS) for 30 min at room temperature. Cells were incubated for 1 hour at room temperature with anti-cytochrome c (Pharmingen, San Jose, Calif.) diluted 1:800 and 15 μg/ml Hoechst 33342 in blocking buffer. Cells were washed 5 times with PBS, and incubated with anti-mouse secondary antibody for 1 hour at room temperature. The cells were washed 5 times with PBS and then mounted in Prolong (Molecular Probes, Eugene, Oreg.).

Cells were scored as apoptotic if cytochrome c appeared diffuse and met the following criteria: 1) presence of diffuse cytochrome c in both the cytoplasm and nucleus; 2) lack of reticulated or punctate staining in the cytoplasm. Typically images were acquired using a 20× objective, and 500-800 cells were scored. For the exact number of cells counted in FIGS. 3A, 4, and 5, see Table 1 below. Nuclei were counted using images of uniformly illuminated Hoechst staining imported into Image J (URL: <http://rsb.info.nih.gov/ij/>).

Significance was determined using unpaired single tailed Student's t-Tests and error bars were calculated using standard deviation.

TABLE 1

| | | | | |
|---|---|---|---|---|
| Day | DMSO | QVD | Act-D + QVD | Act-D |

Diffuse cyt c (%)

| | | | | |
|---|---|---|---|---|
| 1 | 0.0 | 2.0 | 13.0 | 0.8 |
| 2 | 0.4 | 2.3 | 77.0 | 13.4 |
| 3 | 0.8 | 3.2 | 96.2 | 44.2 |

Standard Deviation

| | | | | |
|---|---|---|---|---|
| 1 | 0.0 | 0.4 | 4.2 | 0.9 |
| 2 | 0.7 | 1.0 | 11.0 | 7.7 |
| 3 | 1.2 | 1.3 | 1.8 | 13.2 |

Cells counted

| | | | | |
|---|---|---|---|---|
| 1 | 300 | 767 | 290 | 219 |
| 2 | 435 | 755 | 299 | 84 |
| 3 | 525 | 750 | 256 | 18 |

### EXAMPLE 4

Comparison of Caspase-Independent Cytochrome c Release (CICR) Assay and Standard Apoptotic Assays

A comparison between CICR and standard apoptosis assays showed that the CICR assay is more sensitive. Two of the standard assays, subG1 peak determination and loss of ΔΨm, detected little or no apoptosis in MEFs treated with Act-D for one day (FIGS. 2A and B).

PI exclusion assay. 1×10⁵ cells were treated with Act-D (40 nM in DMSO), DMSO (0.2%), QVD (40 μM), or QVD plus Act-D for 1, 2, or 3 days. QVD (and Act-D+QVD) was replaced each day. At the end of the experiment, PI was added to a final concentration of 10 μg/ml. Following 10 min in the incubator, media were collected and the cells were washed with PBS. The PBS was also collected and combined with the media. The remaining cells were trypsinized and then combined with the mix of PBS and media. The cells were centrifuged at 200 g for 10 minutes and resuspended in 300 μl of PBS. The cells were then quantified and analyzed as above (Cotter and Martin, 1996).

However, the CICR assay showed 13% of the cells were apoptotic at that time point (FIG. 3A). Data from the standard assays could be interpreted to mean that Act-D starts killing MEFs between 24 and 48 hours after exposure. However, the more sensitive CICR assay shows that Act-D begins killing cells within the first 24 hours. Further, the standard PI exclusion assay indicated that only 74% of cells treated with Act-D had died after 3 days, while the CICR assay indicated that 96% of the cells were dead or dying after. 3 days (FIGS. 2C and 3A). We conclude that the CICR assay scores a higher incidence of apoptosis because cytochrome c release kinetically precedes nuclear fragmentation and PI positivity. Additionally, conventional assays could not measure apoptotic cells that had completed the apoptotic program and disintegrated.

### EXAMPLE 5

Sensitivity of the CICR Assay

To further explore the sensitivity of the CICR assay, the background level of apoptosis was measured in an untreated MEF population. It was reasoned that if the assay was efficacious at detecting cumulative apoptotic events, it should detect a progressively increasing number of apoptotic cells over time. Indeed, the CICR assay allowed one to follow the low level of background apoptosis cumulatively and progressively for 3 days in two individual MEF cultures, after which about 2-3% of the cells were dead or dying (FIG. 3B). None of the conventional assays used could reliably measure these low levels of apoptosis (FIG. 2A-C). Moreover, the conventional assays were incapable of measuring the accumulation of apoptotic cells over 3 days (FIG. 2A-C).

### EXAMPLE 6

Using the CICR Assay to Test X-Irradiation Affect on Apoptosis

The best characterized response of fibroblasts to X-irradiation or radiomimetics is induction of a stable permanent growth arrest known as senescence (DiLeonardo et al., 1994). However, these agents are known to induce apoptosis in other cell types, such as lymphocytes (Boreham et al., 1996). It is thought that Bcl-2 protein family members that reside at the mitochondria may be responsible for regulating cell type-specific differences in the response to identical stimuli (Bates and Vousden, 1996). Indeed, different cell types have different ratios of pro- to anti-apoptotic Bcl-2 family members, which is known to affect the susceptibility to apoptosis (Oltvai et al., 1993). Furthermore, inactivation of the anti-apoptotic protein Bcl-x by deamination has been shown to dramatically increase the rate of fibroblast cell death in response to X-irradiation (Deverman et al., 2002).

X-irradiation is known to induce a senescent growth arrest in cultured fibroblasts (DiLeonardo et al., 1994). To further test the sensitivity of the CICR assay, it was asked whether X-irradiation also induced apoptosis, albeit in a small fraction of cells. Human fibroblasts were X-irradiated (10 Gy), then QVD was immediately added to the culture medium, then used the CICR assay to detect apoptosis over the next 3 days. The assay showed that apoptotic cells remained rare in the irradiated population but rising about 1% in the 3 days following radiation (FIG. 4). The finding, using the CICR assay, that X-irradiation causes a very small increase in apoptosis in fibroblasts support the idea that the senescence response predominates when this cell type is irradiated. These results also raise the possibility that the assay detected the small minority of isogenic fibroblasts that have activated the apoptotic response by modifying the activity of Bcl-2 family members.

### EXAMPLE 7

Using the CICR Assay to Determine Background Levels of Apoptosis

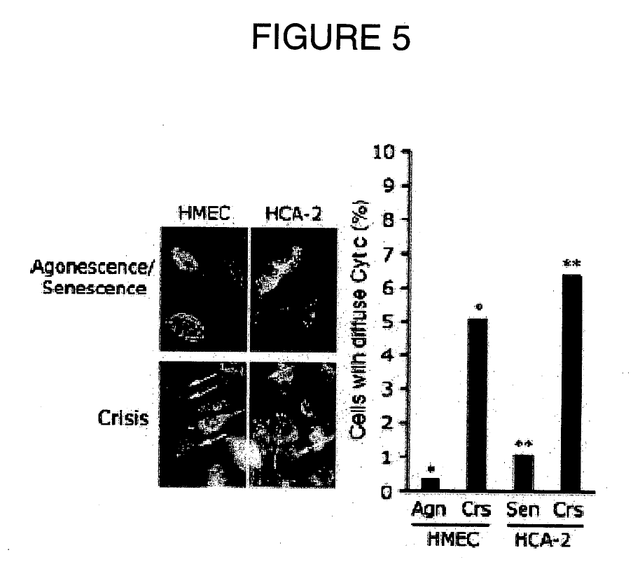

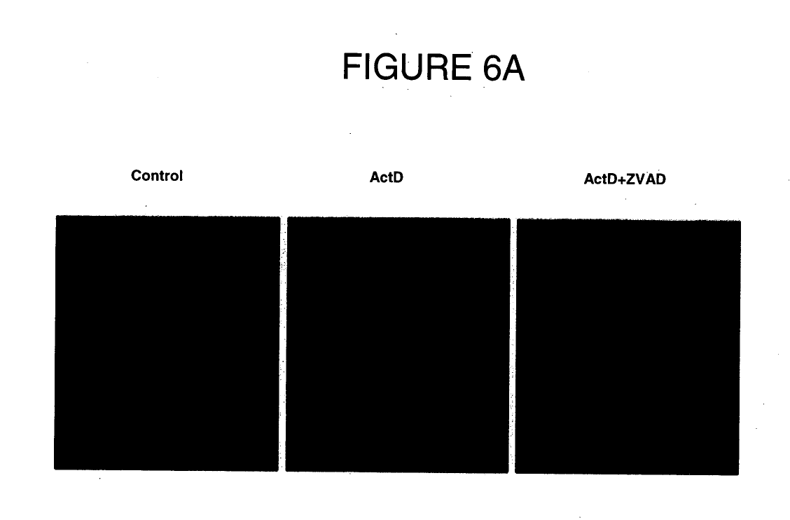

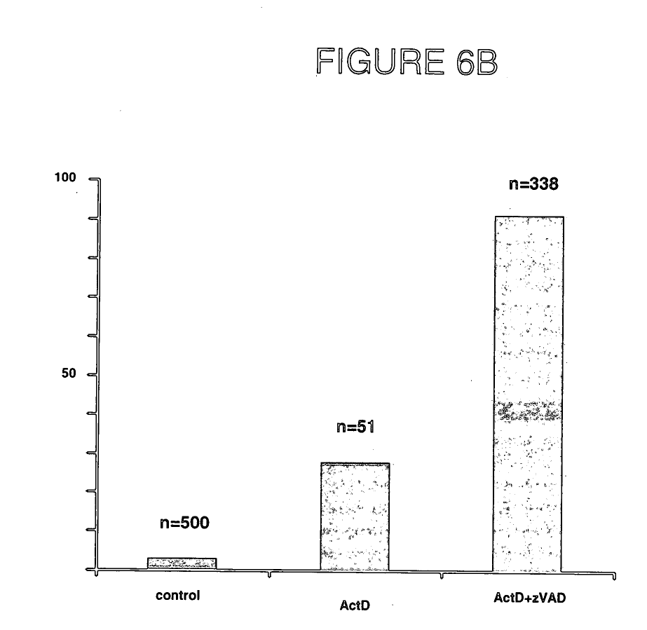

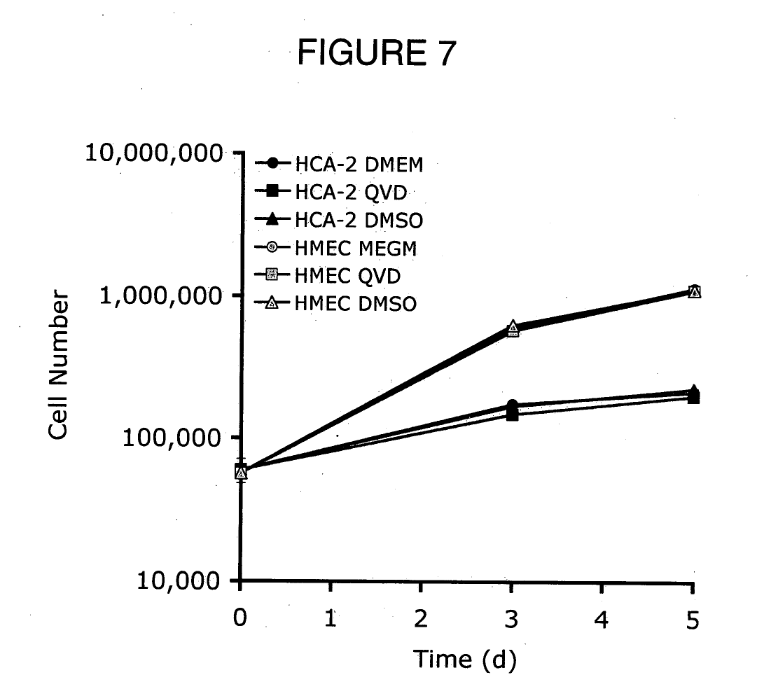

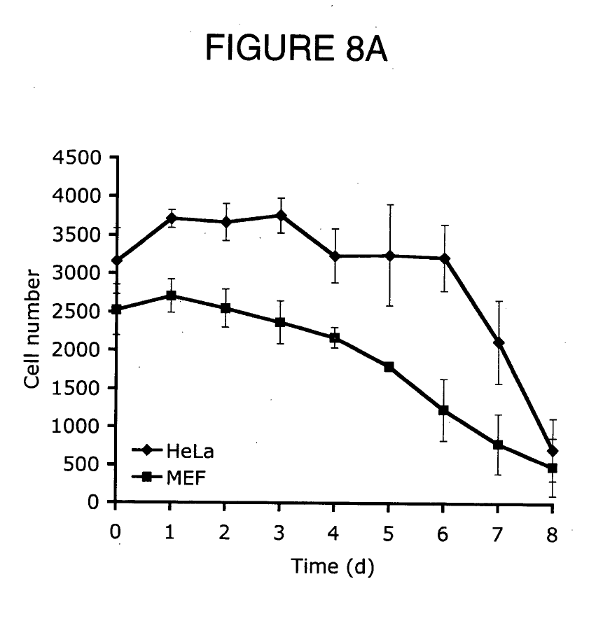

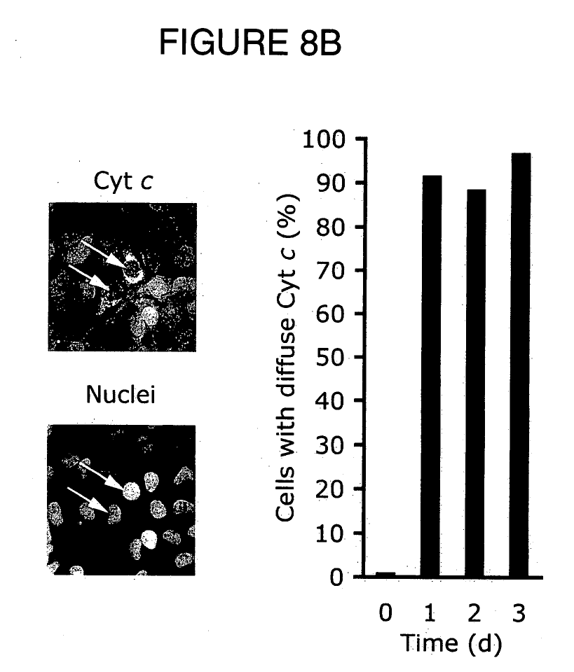

Finally, the CICR assay was used to determine the level of background apoptosis in cultured HMECs. These cells undergo a telomere length-dependent replication block known as agonescence (Romanov et al., 2001), which can be overcome by loss of p53 function (Stampfer et al., 2003). Populations of cells that have short telomeres and defective p53 function eventually enter a state known as crisis, in which cell death occurs gradually but eventually in most cells in the population. In very rare cases, replicatively immortal cells may emerge from cultures in crisis (Shay et al., 1993a). Although many cells die during crisis, it is not known whether they die by apoptosis. To probe for answer to this, the 3 day CICR assay was used to measure the level of apoptosis in cultures that either arrested proliferation at agonescence or had entered crisis owing to inactivation of p53 by the genetic suppressor element GSE22 (Ossovskaya et al., 1996). Over 3 days, apoptosis occurred in <1% of agonescent HMECs and ˜1% of senescent fibroblasts (FIG. 5.) The results are consistent with a report that found that apoptosis is rare at agonescence, as measued by Annexin V binding (Romanov et al., 2001). As expected, these cultures had very few proliferating cells, as determined by BrdU incorporation during the 3 day interval (5.6% and 5% for agonescent and senescent cultures, respectively). By contrast, ˜5% of HMECs and HCA-2 cells in crisis underwent apoptosis over 3 days (FIG. 5.) Consistent with these cultures being in crisis (Wei and Sedivy, 1999), 25% of HMECs and 23% of HCA-2 incorporated BrdU during the 3 day interval, but cell number did not increase. The low rate of apoptosis that was detected during crisis could explain the gradual disappearance of cells from cultures in crisis, but does not rule out other possible modes of cell death, such as necrosis and mitotic catastrophe.

### EXAMPLE 8

Using the CICR Assay to Determine Apoptosis Levels In Vivo

Low rates of apoptosis are hard to reliably measure in tissue culture models. However, detecting any apoptosis in an animal is considerably more difficult. This is because apoptotic cells are rapidly and efficiently engulfed and digested by cells dedicated to the removal of apoptotic cells, such as macrophages, or by neighboring cells.

There are presently few assays designed to detect apoptosis in animals. By far the most popular apoptosis assay for in vivo study is called the Tunel assay, which measures DNA fragmentation that occurs during apoptosis. Like most assays, TUNEL has some limitations so another assay would be quite valuable to the field.

It is contemplated that the CICR assay could also be used in vivo. Just as it was demonstrated in tissue culture conditions, blocking caspases should preserve apoptotic cells in vivo. This should block engulfment and digestion of apoptotic cells and allow apoptotic cells to accumulate. This would make CICR more sensitive than TUNEL.

Caspase-independent cytochome c localization could be measured in tissue sections by immunohistochemistry using an antibody to cytochrome c. The cytochrome c antibody used, 6H2.B4, from BD Bioscience, has not been tested for immunohistochemistry, so its efficacy is unknown. However, cytochrome c antibodies (A-8) from Santa Cruz Biotechnology are advertised to be compatible with immunohistochemistry in paraffin sections.

One potential problem might be that the caspase inhibitor used, such as QVD, would be toxic and kill an animal outright. However, this does not seem to be the case. For example, in contrast to other caspase inhibitors, such as zVAD-fmk, according to the distributors (MP Biomedicals), QVD can be administered safely to an animal at a concentration of 1 g/kg of body weight. This is about 1.9 mM which is nearly 50 fold more concentrated than the concentration we used in tissue culture. Additionally, a group has reported safely using a concentration of 20 mg/kg of body weight, which is about the same concentration used in the Examples above. See Yang L, Sugama S, Mischak R P, Kiaei M, Bizat N, Brouillet E, Joh T H, Beal M F. A novel systemically active caspase inhibitor attenuates the toxicities of MPTP, malonate, and 3NP in vivo. Neurobiol Dis. 2004 November; 17(2):250-9.

In summary, the CICR assay of Example 3, should work in vivo in a mammalian subject. This would be beneficial for a myriad of mouse disease model systems where apoptosis plays a role, including models for degenerative diseases (muscle, brain, etc.). It is also contemplated that the present method potentially could be used to measure the efficacy of chemotherapy in patients in vivo.

While the present compositions and processes have been described with reference to specific details of certain exemplary embodiments thereof, it is not intended that such details be regarded as limitations upon the scope of the invention. The present examples, methods, procedures, specific compounds and molecules are meant to exemplify and illustrate the invention and should in no way be seen as limiting the scope of the invention. Any patents or publications mentioned in this specification and below are indicative of levels of those skilled in the art to which the invention pertains and are hereby incorporated by reference to the same extent as if each was specifically and individually incorporated by reference.

### REFERENCES

Artandi, S. E., and DePinho, R. A. (2000). Mice without telomerase: what can they teach us about human cancer? Nature Med 6, 852-855.
Ashkenazi, A., and Dixit, V. M. (1998). Death receptors: signaling and modulation. Science 281, 1305-1308.
Bates, S., and Vousden, K. H. (1996). p53 in signaling checkpoint arrest or apoptosis. Curr Opin Genet Dev 6, 12-18.
Boreham, D. R., Gale, K. L., Maves, S. R., Walker, J. A., and Morrison, D. P. (1996). Radiation-induced apoptosis in human lymphocytes: potential as a biological dosimeter. Health Phys 71, 685-691.
Campisi, J. (2001). Cellular senescence as a tumor-suppressor mechanism. Trends Cell Biol 11, 27-31.
Campisi, J. (2003a). Cancer and ageing: Rival demons? Nature Rev Canc 3, 339-349.
Campisi, J. (2003b). Cellular senescence and apoptosis: how cellular responses might influence aging phenotypes. Exp Gerontol 38, 5-11.
Caserta, T. M., Smith, A. N., Gultice, A. D., Reedy, M. A., and Brown, T. L. (2003). Q-VD-OPh, a broad spectrum caspase inhibitor with potent antiapoptotic properties. Apoptosis 8, 345-352.
Chang, S., Multani, A. S., Cabrera, N. G., Naylor, M. L., Laud, P., Lombard, D., Pathak, S., Guarente, L., and DePinho, R. A. (2004). Essential role of limiting telomeres in the pathogenesis of Werner syndrome. Nat Genet 36, 877-882.
Cotter, T. G., and Martin, S. J. (1996). Techniques in Apoptosis: A User's Guide (London, Portland Press).
de Boer, J., Andressoo, J. O., de Wit, J., Huijmans, J., Beems, R. B., van Steeg, H., Weeda, G., van der Horst, G. T., van Leeuwen, W., Themmen, A. P., et al. (2002). Premature aging in mice deficient in DNA repair and transcription. Science 296, 1276-1279.
Deverman, B. E., Cook, B. L., Manson, S. R., Niederhoff, R. A., Langer, E. M., Rosova, I., Kulans, L. A., Fu, X., Weinberg, J. S., Heinecke, J. W, et al. (2002). Bcl-xL deamidation is a critical switch in the regulation of the response to DNA damage. Cell 111, 51-62.
DiLeonardo, A., Linke, S. P., Clarkin, K., and Wahl, G. M. (1994). DNA damage triggers a prolonged p53-dependent G1 arrest and long-term induction of Cip1 in normal human fibroblasts. Genes Dev 8, 2540-2551.
Enari, M., Sakahira, H., Yokoyama, H., Okawa, K., Iwamatsu, A., and Nagata, S. (1998). A caspase-activated DNase that degrades DNA during apoptosis, and its inhibitor ICAD. Nature 391, 43-50.
Green, D. R., and Evan, G. I. (2002). A matter of life and death. Cancer Cell 1, 19-30.
Hammond, S. L., Ham, R. G., and Stampfer, M. R. (1984). Serum-free growth of human mammary epithelial cells: rapid clonal growth in defined medium and extended serial passage with pituitary extract. Proc Natl Acad Sci USA 81, 5435-5439.
Hara, E., Tsurui, H., Shinozaki, A., Nakada, S., and Oda, K. (1991). Cooperative effect of antisense-Rb and antisense-p53 oligomers on the extension of life span in human diploid fibroblasts, TIG-1. Biochem Biophys Res Commun 179, 528-534.
Hasty, P., Campisi, J., Hoeijmakers, J., van Steeg, H., and Vijg, J. (2003). Aging and genome maintenance: lessons from the mouse? Science 299, 1355-1359.
Jacobson, M. D., Well, M., and Raff, M. C. (1997). Programmed cell death in animal development. Cell 88, 347-354.
Jejurikar, S. S., and Kuzon, W. M., Jr. (2003). Satellite cell depletion in degenerative skeletal muscle. Apoptosis 8, 573-578.
Joaquin, A. M., and Gollapudi, S. (2001). Functional decline in aging and disease: A role for apoptosis. J Am Geriatr Soc 49, 1234-1240.
Koopman, G., Reutelingsperger, C. P., Kuijten, G. A., Keehnen, R. M., Pals, S. T., and van Oers, M. H. (1994). Annexin V for flow cytometric detection of phosphatidylserine expression on B cells undergoing apoptosis. Blood 84, 1415-1420.
Martin, S. J., Reutelingsperger, C. P., McGahon, A. J., Rader, J. A., van Schie, R. C., LaFace, D. M., and Green, D. R. (1995). Early redistribution of plasma membrane phosphatidylserine is a general feature of apoptosis regardless of the initiating stimulus: inhibition by overexpression of Bcl-2 and Abl. J Exp Med 182, 1545-1556.
Meier, P., Finch, A., and Evans, G. (2000). Apoptosis in development. Nature 407, 796-801.
Murakami, S., Salmon, A., and Miller, R. A. (2003). Multiplex stress resistance in cells from long-lived dwarf mice. Faseb J 17, 1565-1566.
Nishimura, E. K., Granter, S. R., and Fisher, D. E. (2005). Mechanisms of hair graying: incomplete melanocyte stem cell maintenance in the niche. Science 307, 720-724.
Oltvai, Z. N., Milliman, C. L., and Korsmeyer, S. J. (1993). Bcl-2 heterodimerizes in vivo with a conserved homolog, Bax, that accelerates programmed cell death. Cell 74, 609-619.
Ormerod, M. G., Collins, M. K., Rodriguez-Tarduchy, G., and Robertson, D. (1992). Apoptosis in interleukin-3-dependent haemopoietic cells. Quantification by two flow cytometric methods. J Immunol Methods 153, 57-65.
Ossovskaya, V. S., Mazo, I. A., Chemov, M. V., Chemova, O. B., Strezoska, Z., Kondratov, R., Stark, G. R., Chumakov, P. M., and Gudkov, A. V. (1996). Use of genetic suppressor elements to dissect distinct biological effects of separate p53 domains. Proc Natl Acad Sci USA 93, 10309-10314.
Parrinello, S., Samper, E., Krtolica, A., Goldstein, J., Melov, S., and Campisi, J. (2003). Oxygen sensitivity severely limits the replicative lifespan of murine fibroblasts. Nat Cell Biol 5, 741-747.
Ricci, J. E., Munoz-Pinedo, C., Fitzgerald, P., Bailly-Maitre, B., Perkins, G. A., Yadava, N., Scheffler, I. E., Ellisman, M. H., and Green, D. R. (2004). Disruption of mitochondrial function during apoptosis is mediated by caspase cleavage of the p75 subunit of complex I of the electron transport chain. Cell 117, 773-786.
Romanov, S. R., Kozakiewicz, B. K., Holst, C. R., Stampfer, M. R., Haupt, L. M., and Tlsty, T. D. (2001). Normal human mammary epithelial cells spontaneously escape senescence and acquire genomic changes. Nature 409, 633-637.
Sahara, S., Aoto, M., Eguchi, Y., Imamoto, N., Yoneda, Y., and Tsujimoto, Y. (1999). Acinus is a caspase-3-activated protein required for apoptotic chromatin condensation. Nature 401, 168-173.
Sharpless, N. E., and DePinho, R. A. (2004). Telomeres, stem cells, senescence, and cancer. J Clin Invest 113, 160-168.
Shay, J. W., Pereira-Smith, O. M., and Wright, W. E. (1991). A role for both RB and p53 in the regulation of human cellular senescence. Exp Cell Res 196, 33-39.
Shay, J. W., Van Der Haegen, B. A., Ying, Y., and Wright, W. E. (1993a). The frequency of immortalization of human fibroblasts and mammary epithelial cells transfected with SV40 large T-antigen. Exp Cell Res 209, 45-52.
Shay, J. W., Van Der Haegen, B. A., Ying, Y., and Wright, W. E. (1993b). The frequency of immortalization of human fibroblasts and mammary epithelial cells transfected with SV40 large T-antigen. Exp Cell Res 209, 45-52.
Shay, J. W., and Wright, W. E. (2004). Senescence and immortalization: role of telomeres and telomerase. Carcinogenesis.
Stampfer, M. R., Garbe, J., Nijjar, T., Wigington, D., Swisshelm, K., and Yaswen, P. (2003). Loss of p53 function accelerates acquisition of telomerase activity in indefinite lifespan human mammary epithelial cell lines. Oncogene 22, 5238-5251.
Suh, Y., Lee, K. A., Kim, W. H., Han, B. G., Vijg, J., and Park, S. C. (2002). Aging alters the apoptotic response to genotoxic stress. Nature Med 8, 3-4.
Thomberry, N. A., and Lazebnik, Y. (1998). Caspases: enemies within. Science 281, 1312-1316.
Tyner, S. D., Venkatachalam, S., Choi, J., Jones, S., Ghebranious, N., Igelmann, H., Lu, X., Soron, G., Cooper, B., Brayton, C., et al. (2002). p53 mutant mice that display early ageing-associated phenotypes. Nature 415, 45-53.
Vaux, D. L., and Korsmeyer, S. J. (1999). Cell death in development. Cell 96, 245-254.
Walker, N. I., Harmon, B. V., Gobe, G. C., and Kerr, J. F. (1988). Patterns of cell death. Methods Achiev Exp Pathol 13, 18-54.
Waterhouse, N. J., Goldstein, J. C., von Ahsen, O., Schuler, M., Newmeyer, D. D., and Green, D. R. (2001). Cytochrome c maintains mitochondrial transmembrane potential and ATP generation after outer mitochondrial membrane permeabilization during the apoptotic process. J Cell Biol 153, 319-328.
Wolf, B. B., and Green, D. R. (1999). Suicidal tendencies: apoptotic cell death by caspase family proteinases. J Biol Chem 274, 20049-20052.
Zhang, Y., and Herman, B. (2002). Ageing and apoptosis. Mech Ageing Dev 123, 245-260.

What is claimed is:

1. A method of detecting and monitoring apoptosis is comprised of the following steps: (1) providing a cell sample, (2) treating the cells with a caspase inhibitor for an extended period of time, (3) washing the cells, (4) treating the cells with an antibody to a caspase-independent signaling protein to stain apoptotic cells and (5) detecting apoptotic cells.

2. The method of claim 1, further comprising the step of treating the cells with a stain or dye to detect chromatin condensation, phosphatidylserine exposure, or nuclear fragmentation.

3. The method of claim 1, wherein the caspase inhibitor is a general caspase inhibitor or a specific inhibitor to Caspase 1, 2, 3, 4, 5, 6, 7, 8, 9, 10 or 13.

4. The method of claim 3, wherein the caspase inhibitor is selected from the group consisting of Quinoline-Val-Asp-Ch₂-O-Ph (QVD), zVAD or BAF.

5. The method of claim 1, wherein the extended period of time is at least 3 days.

6. The method of claim 1, wherein the caspase-independent signaling protein is a pro-apoptotic Bcl-2 famiiy member, such as Bax, Bad, Bim, Puma, Noxa, or a mitochondrial protein that is released into the cytoplasm, such as apoptosis protease activating factor-1 (APAF-1), Cytochrome c, SMAC/Diablo, Omi/HtrA2, endonucleaseG, and apoptosis inducing factor (AIF).

7. The method of claim 1, further comprising the step of treating the cells with a secondary antibody conjugated to a fluorescent probe after step 4 to amplify the antibody staining signal.

8. The method of claim 7, wherein the apoptotic cells are detected by the amount of fluorescence.

9. The method of claim 1, further comprising a step of treating the cells with a stain or dye to detect a marker of apoptosis such as, chromatin condensation, phosphatidylserine exposure or nuclear fragmentation.

10. A kit to carry out the method of claim 1, comprising a set of vials or containers containing the necessary compounds, reagents, inhibitors, antibodies, dyes and buffers formulated and ready for use and instructions for carrying out said method.
